(19) United States
(12) Patent Application Publication
(10) Pub. No.: US 2003/0207819 A1
(43) Pub. Date: Nov. 6, 2003

(54) Compositions and methods for treating inflammatory connective tissue diseases

(76) Inventor: Roland W. Moskowitz, Pepper Pike, OH (US)

Correspondence Address: Barbara E. Arndt, Ph.D., Jones Day, North Point, 901 Lakeside Avenue, Cleveland, OH, 44114, US

(21) Appl. No.: 10/428,265

(22) Filed: May 2, 2003

Related U.S. Application Data
(60) Provisional application No. 60/377,281, filed on May 2, 2002.

(51) Int. Cl. A61K 31/7048

(52) U.S. Cl. 514/28; 514/29

(57) ABSTRACT

The invention provides compositions and methods utilizing 14- and 15-member macrolide antibiotics for the treatment of patients with connective tissue diseases. The methods of the invention provide for the administration to a patient of a therapeutically effective amount of a 14-member macrolide antibiotic, a 15-member macrolide antibiotic, pharmaceutically acceptable derivatives thereof, and combinations thereof for a period of time sufficient to obtain a desired alleviation of one or more symptoms of the connective tissue disease.

This application claims the benefit of U.S. Ser. No. 60/377,281 filed May 2, 2002, which disclosure is incorporated herein by reference.

### BACKGROUND OF THE INVENTION

Although the exact etiology of most inflammatory rheumatic and other chronic inflammatory connective tissue diseases is uncertain, it is known that immunoregulatory abnormalities can lead to vascular and cellular injury from the action of leukocytes and the mediators of inflammation that they produce. The accumulation of these cells is mediated by the same mechanisms involved in the normal response to bacterial invasion.

Abnormal regulation of cytokine production by leukocytes, manifested by overproduction of inflammatory cytokines and/or decreased production of anti-inflammatory cytokines, can play a key role in the pathogenesis of immune-mediated diseases. For example, rheumatoid arthritis is characterized by increased levels of the inflammatory cytokines IL-1β, TNFα, and IL-6, and decreased levels of the anti-inflammatory cytokine, IL-10. Similar cytokine profiles can be found in other inflammatory arthritides. Adhesion molecules (cell surface molecules essential to cell-to-cell interaction during immune activation and cell migration and recruitment) such as ICAM- 1, VCAM- 1, LFA- 1, E-selectin, and the like, are involved to various degrees in the pathogenesis of many rheumatic diseases including rheumatoid arthritis, giant cell arteritis, psoriatic arthritis and others. The release of nitric oxide, superoxide radicals, and other toxic oxygen metabolites (the respiratory burst) by neutrophils, disordered apoptosis (programmed cell death), and production of prostaglandins can also contribute to tissue injury.

Current medically approved therapies available for inflammatory rheumatic diseases either non-specifically inhibit cytokine production and other leukocyte functions (e.g., corticosteroids, methotrexate, alkylating agents, cyclosporine, and the like) or specifically inhibit cytokines (e.g., infliximab, etanercept, anakinra, and the like). Non-steroidal anti-inflammatory drugs (NSAIDs) decrease the production of prostaglandins that are produced by the stimulation of the COX-2 enzyme by TNFα and IL-1. Unfortunately, not all patients respond to these agents or they are troubled by toxicity or side effects that are potentially significant with all of these agents.

The 14- and 15-member macrolide family of antibiotics (e.g., erythromycin, clarithromycin, azithromycin, and others) has been used for decades for the treatment of a variety of infections. These antibiotics have a broad spectrum of antibacterial activity against both gram positive and intracellular organisms with good tissue penetration. Interest in the potential immunomodulatory effects of the macrolide derivatives of erythromycin A in pulmonary disease was aroused when it was shown that long term treatment with erythromycin and troleandomycin improved the clinical status of steroid dependent patients with asthma. Subsequent reports of improvement of diffuse panbronchiolitis (DPB) with long term macrolide therapy, i.e., erythromycin and clarithromycin, prompted renewed interest in this macrolide family of antibiotics as possible anti-inflammatory or immunomodulatory agents. Evidence suggests that macrolides may have an immunomodulatory effect in airway diseases such as asthma, DPB, chronic sinusitis and a variety of viral and bacterial infections, although the mechanism of action in these diseases has not been elucidated.

However, other than their clinical use in the long term treatment of diffuse DPB, 14- and 15-member macrolide antibiotics have not been suggested for the possibility of anti-inflammatory/immunomodulatory effects in the clinical treatment of the inflammatory rheumatic and other chronic inflammatory connective tissue diseases.

### SUMMARY OF THE INVENTION

It has unexpectedly been discovered that 14- and 15-member macrolide antibiotics, such as erythromycin A, clarithromycin, roxithromycin, azithromycin, dirithromycin, HMR 3004, and the like, can successfully be used for long-term adjuvant therapy in chronic inflammatory rheumatic connective tissue diseases. The invention further encompasses the use of these macrolide antibiotics for treatment of other inflammatory diseases including, but not limited to, rheumatoid arthritis, psoriatic arthritis, giant cell arteritis, systemic lupus erythematosus, polymyalgia rheumatica, polymyositis, inflammatory bowel disease, and the like. It has unexpectedly been discovered that treatment with these antibiotics over a period of time sufficient to produce an immunosuppressive effect alleviates one or more symptoms of connective tissue disease in a significant number of treated patients. It was also discovered that the macrolide treatment can alleviate symptoms in a dose-dependent fashion.

One embodiment of the invention provides a composition for treating a patient having a connective tissue disease the composition comprising a therapeutically effective amount of a macrolide antibiotic selected from the group consisting of a 14-member macrolide, a 15-member macrolide, pharmaceutically acceptable derivatives thereof, and combinations thereof.

Another embodiment of the invention provides a method for treating a patient having a connective tissue disease, the method comprising administering to patient a therapeutically effective amount of a macrolide antibiotic selected from the group consisting of a 14-member macrolide, a 15-member macrolide, pharmaceutically acceptable derivatives thereof, and combinations thereof Preferably, the therapeutically effective amount of the antibiotic is administered to the patient for a period of time sufficient to alleviate at least one symptom of the connective tissue disease.

In a further embodiment, the method includes the additional step of readministering a therapeutically effective dose at an interval as required to maintain the desired alleviation of one or more symptoms of connective disease.

The macrolide antibiotics according to the embodiments of the invention can be natural, synthetic, semi-synthetic, or mixtures thereof Preferably, the macrolide antibiotics according to the above embodiments of the invention are selected from the group consisting of erythromycin A, clarithromycin, roxithromycin, dirithromycin, azithromycin, HMR 3004, pharmaceutically acceptable derivatives thereof, and combinations thereof

### DETAILED DESCRIPTION OF THE INVENTION

Compositions and methods for the treatment of patients having a connective tissue disease are provided by the present invention. Embodiments of compositions comprise a therapeutically effective amount of a macrolide antibiotic selected from the group consisting of a 14-member macrolide antibiotic, a 15-member macrolide antibiotic, pharmaceutically acceptable derivatives thereof and combinations thereof According to the methods of treatment in the embodiments of the invention, connective tissue diseases are treated in a patient such as a human or other mammal by administering to the patient a therapeutically effective amount of a 14- or 15-member macrolide antibiotic, and salts, derivatives or combinations thereof, optionally in a pharmaceutically acceptable carrier, in such amounts and for such time as is necessary to achieve the desired result.

By a “therapeutically effective amount” of the macrolide antibiotic is meant a sufficient amount of the antibiotic to alleviate at least one symptom of the disease at a reasonable benefit/risk ratio applicable to any medical treatment. The therapeutically effective amount of the antibiotic, however, can be determined by the practitioner within the scope of sound medical judgment. For example, the therapeutically effective amount can be a normal clinical dose used to treat bacterial infections. As a further non-limiting example, the therapeutically effective amount can be a maximum dose capable of being safely received by the patient. As a further non-limiting example, the therapeutically effective amount can be a clinically effective dose capable of alleviating at least one symptom of a connective tissue disease. The specific therapeutically effective dose level for any particular patient will depend upon a variety of factors including the disorder being treated and the severity of the disorder; the activity of the specific antibiotic employed; the specific pharmaceutical composition employed; the age, body weight, general health, sex and diet of the patient, the time of administration, route of administration, and rate of excretion of the specific antibiotic employed; the duration of the treatment; drugs used in combination or coincidental with the specific antibiotic employed; and like factors well known in the medical arts.

The macrolide antibiotics of the present invention can be used in combination with other drugs used in the treatment of connective tissue diseases. For example, the macrolide antibiotics can be used in combination with drugs such as, but not limited to, auranofin, azathioprine, cyclophosphamide, cyclosporine, etanercept, hydroxychloroquine, inflaximab, leflunomide, methotrexate, minocycline, mycophenalate mofetil, penicillamine, sulfasalazine, tacrolimus, and the like.

The 14- and 15-member macrolide antibiotics of the present invention include pharmaceutically acceptable derivatives of those compounds, which can be prepared using conventional, well known preparative techniques. By “pharmaceutically acceptable derivatives” is meant pharmaceutically acceptable salts, esters and other derivatives now known or developed in the future. Particular salts can be obtained by treating a 14- or 15-member macrolide antibiotic with a corresponding acid. Salts of the macrolide antibiotics of the invention include those formed in reaction with inorganic and organic acids including, but not limited to, mineral acids such as, but not limited to, hydrochloric acid, hydrobromic acid, hydroiodic acid, sulfuric acid, phosphoric acid, and the like; organic acids such as, but not limited to, acetic acid, propionic acid, trifluoroacetic acid, maleic acid, tartaric acid, methanesulfonic acid, benzenesulfonic acid, p-toluenesulfonic acid, citric acid, stearic acid, succinic acid, aminoethanesulfonic acid, ethylsuccinic acid, laurylsulfuric acid, and the like; and amino acids such as, but not limited to, aspartic acid, glutamic acid, and the like.

The period of time over which a macrolide antibiotic is administered to any particular patient to produce an alleviation of at least one symptom of connective tissue disease will also depend on the variety of factors mentioned above in reference to the therapeutically effective dose level. For example, in some patients, a clinical effect can be observed within about two weeks. However, based on the general chronicity of the diseases responding to these agents, it is preferable that the period of administration be long term and continuous. In certain situations, short-term periodic administration, such as about three months to about six months, may be desired. With any treatment period the therapeutically effective dose can be readministered at an interval as required to obtain and/or maintain the desired alleviation of one or more symptoms of connective tissue disease. For example, the antibiotic can be administered at time intervals that are not consecutive days, such as, but not limited to, every other day, every three days, once a week, or the like, especially if the antibiotic has a long-acting activity. Further, the antibiotic can be administered on a varying dosage schedule, such as, but not limited to, one dosage level on day one and reduced or otherwise varying dosage levels on subsequent days until the treatment period ends or the administration cycle begins again. The therapeutically effective dosage levels of the antibiotic in the above described administration cycles can be determined by routine experimentation.

The antibiotic or antibiotics can be administered to a patient by any route including, but not limited to, oral, transdermal, intravenous, intradermal, intraperitoneal, subcutaneous, intramuscular, cerebrospinal, cerebrospinal, topical, or combinations of these, or the like.

Compositions of the present invention can be made by formulating a therapeutically effective amount of a 14- or 15-member macrolide antibiotic of the invention together with a pharmaceutically acceptable carrier for parenteral injection, oral administration in solid or liquid form, rectal administration, topical and the like. The total daily dose of the antibiotic administered to a human or other mammal by the methods of the invention in single or in divided doses can be in amounts, for example, from about 0.01 mg/kg to about 1000 mg/kg of body weight/day, preferably about 0.01 mg/kg to about 500 mg/kg, more preferably about 0.01 mg/kg to about 250 mg/kg of body weight/day, especially about 0.01 mg/kg to about 100 mg/kg, and more especially about 0.01 to about 25 mg/kg body weight, without limitation. As a further non-limiting example, the total daily dose of erythromycin administered to a patient in a single or divided doses can be about 10 mg/kg to about 75 mg/kg, preferably about 57 mg/kg. As a further non-limiting example, the total daily dose of clarithromycin administered to a patient in a single or divided doses can be about 5 mg/kg to about 30 mg/kg, preferably about 14 mg/kg. As a further non-limiting example, the total daily dose of azithromycin administered to a patient in a single or divided doses can be about 1 mg/kg to about 30 mg/kg, preferably about 4 mg/kg. Similarly, the non-limiting total daily dose of dithromycin administered to a patient in single or divided doses can be about 1 mg/kg to about 20 mg/kg, preferably about 7 mg/kg. Single dose compositions can contain such amounts or submultiples thereof to make up the daily dose. Typically, treatment regimens according to the invention methods comprise administration to a patient in need of such treatment from about 1 mg to about 5500 mg of the antibiotic per day in single or multiple doses.

The amount of active ingredient that can be combined with a pharmaceutically acceptable carrier materials to produce a single dose form will vary depending upon the patient treated and the particular mode of administration. Moreover, the effective amount of an antibiotic of the present invention can vary with the particular connective tissue disease being treated; the severity of the disease; the duration of the treatment; the specific antibiotic, ester, or salt being employed; the age and weight of the patient; and like factors well known to those of skill in the medical arts. In addition to the following described pharmaceutical compositions, the therapeutic composition can be formulated as polymeric compositions, inclusion complexes, such as cyclodextrin inclusion complexes, liposomes, microspheres, microcapsules, and the like, without limitation.

Compositions for parenteral injection can comprise pharmaceutically acceptable sterile aqueous or non-aqueous solutions, suspensions, or emulsions. Examples of suitable non-aqueous carriers, diluents, solvents, or vehicles include, but are not limited to, propylene glycol, polyethylene glycol, vegetable oils, and injectable organic esters such as ethyl oleate. Such compositions can also contain adjuvants such as preserving, wetting, emulsifying, dispersing agents and other common known ingredients, and the like. These solutions are suitable for intravenous, intramuscular, subcutaneous, and intraperitoneal administration. The compositions can be sterilized, for example, by filtration through a bacteria-retaining filter, or by incorporating sterilizing agents into the compositions. The compositions can be manufactured in the form of sterile solid compositions that can be dissolved in sterile water, or some other sterile injectable medium immediately before use. The sterile media employed are all readily available.

Solid dosage forms for oral administration include capsules, tablets, pills, powders, and granules. The compositions can, if desired, contain additional ingredients including, but not limited to, flavorings, binders, lubricating agents, disintegrants and excipients. Binding agents can include, but are not limited to, polyvinylpyrrolidone, sucrose, lactose, gelatine, and acacia. Lubricating agents can include, but are not limited to, magnesium stearate, sodium lauryl sulfate, talc, and the like. Disintegrants can include, but are not limited to, starch, methylcellulose, alginic acid, and certain complex silicates. Excipients can include, but are not limited to, sodium citrate, calcium carbonate, and calcium phosphate. Solid oral preparations can also be prepared with enteric or other coatings that modulate or otherwise control release of the active ingredients.

Liquid dosage forms for oral administration include, but are not limited to, pharmaceutically acceptable emulsions, solutions, suspensions, syrups, and elixirs containing inert nontoxic diluents commonly used in the art. Examples of nontoxic diluents commonly used in the art include, but are not limited to, water, ethanol, propylene glycol, glycerine, and combinations thereof. Such compositions can also contain adjuvants such as, but not limited to, wetting, emulsifying, suspending, sweetening, flavoring, coloring, and perfuming agents.

Topical dosage forms include, but are not limited to, creams, gels, ointments, lotions, powders, pastes, suspensions, sprays, and aerosols. Typically, topical formulations include diluents such as water and alcohol, and one or more thickening agents, emulsifiers, stabilizers, humectants, and/or emollients including, but not limited to, xanthan gum, petrolatum, beeswax, polyethylene glycol, sorbitol, mineral oil, lanolin, squalene, and the like.

The pharmaceutical compositions of the present invention can be prepared by conventional techniques such as those described in standard textbooks of pharmaceutics such as, but not limited to, Remington: The Science and Practice of Pharmacy, 19^{th} Ed., 1995 and the British Pharmacopoeia.

Antibiotics for use in compositions and methods according to embodiments of the invention comprise erythromycin A and derivatives of erythromycin A that contain a 14- or 15-member ring structure. Derivatives of erythromycin A that contain a 14- or 15-member ring structure include, but are not limited to, clarithromycin, roxithromycin, dirithromycin, HMR 3004, azithromycin, and pharmaceutically acceptable derivatives thereof The antibiotic can be natural, synthetic, semi-synthetic, or combinations thereof Multiple antibiotics can be combined for treatment. The chemical structures of these antibiotics are well known and are elucidated, for example, in Jeffrey C. Hoyt & Richard A. Robbins, FEMS Microbiology Letters 205 (2001), 1-7, the disclosure of which pertaining to the chemical structures is hereby incorporated by reference.

Clarithromycin is a semi-synthetic macrolide antibiotic, 6-O-methylerythromycin, as disclosed in U.S. Pat. No. 4,743,593, hereby incorporated by reference. Clarithromycin exhibits excellent antibacterial activity against gram-positive bacteria, some gram-negative bacteria, anaerobic bacteria, Mycoplasma, and Chlamidia. It is stable under acidic conditions and is efficacious when administered orally. A non-limiting exemplary commercially available source of clarithromycin is BIAXIN® from Abbott Laboratories. Clarithromycin is available, for example, as tablets and as an oral suspension. The oral suspension is particularly useful for patients such as children and the elderly who have difficulty swallowing tablets. However, because 6-O-methylerythromycin A has such pronounced bitterness, it may be necessary to provide a modified form of the antibiotic that is more palatable, such as 6-O-methylerythromycin A-carbomer complex, such as that disclosed in U.S. Pat. No. 5,945,405. Accordingly, the compositions and methods of the invention can employ clarithromycin in any non-limiting form, such as pharmaceutically acceptable derivatives thereof.

Roxithromycin is a 14-member macrolide antibiotic derived from erythromycin A currently used as a treatment for cryptosporidiosis and for other infections, including chlamydia, sinusitis, bacterial-associated respiratory disease tract infections, gum infections such as gingivitis, pneumonia, skin infections such as erysipelas, and bacterial infections associated with stomach and intestinal ulcers. A non-limiting exemplary commercially available source of roxithromycin is RULID™ from Albert-Roussel Pharma GmbH.

Azithromycin is a 15-member macrolide antibiotic derived from erythromycin; however, it differs chemically in that a methyl-substituted nitrogen atom is incorporated into the lactone ring. A non-limiting exemplary commercially available source of azithromycin is ZITHROMAX® from Pfizer Laboratories in tablet and oral suspension. This antibiotic is currently recommended as an antibacterial for adults, young adults, and children.

HMR 3004 is a 14-member ketolide antibiotic belonging to a class of semi-synthetic macrolides with activity against pathogens resistant to erythromycin (described in Agouridas et al., 1998, M. Med. Chem. 41: 4080-4100, incorporated herein by reference).

Dirithromycin, also known as 9-deoxo-11-deoxy-9,11-{imino[2-(2-methoxyethoxy)-ethylidene]oxy}-(9S,16R)-erythromycin, is a 14-member macrolide antibiotic derived from erythromycin. This antibiotic is described, for example in Example 9 of U.S. Pat. No. 4,048,306, the disclosure of which related to dirithromycin is hereby incorporated by reference. The spectrum of antimicrobial activity of this antibiotic approximates that of erythromycin; however, dirithromycin has the advantageous property of providing high concentrations of antibiotic activity in the tissues while the plasma levels of the antibiotic remain low (see U.S. Pat. No. 5,635, 613, the disclosure of which relating to dirithromycin is hereby incorporated by reference). A non-limiting exemplary commercially available source of dirithromycin is DYNABAC® from Eli Lilly & Co.

The 14-member and/or 15-member macrolide antibiotics included in the scope of the invention methods are not intended to be limited to the foregoing examples, as all 14-member and 15-member macrolide antibiotics now known or discovered in the future, including pharmaceutically acceptable derivatives, are within the scope of the invention.

### EXAMPLES

The potential utility of macrolide antibiotics in the treatment of connective tissue disease is illustrated in the observation of significant improvement in symptoms of connective tissue disease in four patients. Improvements were observed in the first two patients after they were treated with clarithromycin for an unrelated infection. These two patients have subsequently been placed on long-term clarithromycin therapy with maintenance of the response. The second two patients have also shown improvements in symptoms of connective tissue disease.

### Example 1

A 70 year-old female patient was first seen on Nov. 13, 1998.Symptoms included joint pain with diffuse polyarthralgias. She had a prior history that year of laryngitis, bronchitis and blepharitis with a Bell's palsy. She stated that her fingers felt “swollen”. She also complained of dry eyes for which she used eye drops. She reported that her daughter has a positive “lupus test.” Physical examination revealed no joint swelling, tenderness or limitation. A laboratory study performed at that time revealed a slightly elevated creatine phosphokinase (CPK), a sedimentation rate of 76, a positive anti-nuclear antibody test (ANA) and a negative rheumatoid factor (RF). Subsequent laboratory studies have shown an ANA value of 1-40 and negative, respectively. Lyme antibody titers were negative.

When the patient was subsequently seen in December 1998, she had an upper respiratory infection for which she began taking clarithromycin 500 mg b.i.d. (BIAXIN®, Abbott Laboratories). She felt that her symptoms with respect to connective tissue changes were significantly improved. Her original diagnosis was consistent with undifferentiated connective tissue disease with her slightly positive ANA, dry eyes and polyarthralgias. She felt that the clarithromycin had helped. We elected to continue her on this antibiotic. She said she felt “great on this medication”, 500 mg/bid. When we reduced the dose to 250 mg/bid her symptoms returned, and once again improved essentially completely on 500 mg/bid indicating a dose-dependent response. Because the patient was on Lodine (NSAID) at the same time, we elected to see if she could stop the Lodine and still have relief of symptoms. She had some increase of symptoms off Lodine but subsequently continued to improve off Lodine. Periodic trials of reduction of clarithromycin were associated with increased joint symptoms. At a reduced dose of 750 mg/day she gets increased swelling of the hands, painful wrists, knee swelling and hand stiffness. The patient has continued on clarithromycin, continuing to do well. It is of interest that she was switched to azithromycin (ZITHROMAX®, Pfizer, Inc.), 500 mg on day I and 250 mg/day on days 2 through 5, for “cat scratch infection” and was taken off clarithromycin. She had no increase in symptoms on the azithromycin. When last seen on Feb. 12, 2002, she continued to do well except for some osteoarthritic changes of her hands. Her undifferentiated connective disease was under control with the clarithromycin.

### Example 2

Subsequent to the observations made in Example 1, the patient was re-challenged with clarithromycin, and re-evaluated as to symptomatic efficiency. In parallel with clinical observations, laboratory assessments including erythrocyte sedimentation rate, C-reactive protein, and serum interleukins 1 and 6 were measured. The laboratory results are illustrated in Table 1.

On Jul. 29, 2002 the clarithromycin treatment of the patient from Example 1 was discontinued (Table 1). Her additional medications included: ZESTRIL® (Astra Zenica) at 1 mg/t.i.d., CALTRATE® with Vitamin D (Wyeth)/b.i.d., UNITHROID™ (Jerome Stevens Pharmaceuticals, Inc.) at 0.05 mg/day, TROZODONE® (Watson Laboratories, Inc.) at 50 mg/h.s., TYLENOL® (McNeil Consumer Brands) at 1 gm/p.r.n., multi-vitamins/p.r.n., iron tablets, quinine at 260 mg, and non-specific herbs. At the time clarithromycin was discontinued her symptoms were described as being in good control. When the patient was observed on Aug. 8, 2002, 10 days off clarithromycin, she complained of generalized joint pain, associated with swelling. The symptoms were sufficiently severe that she asked to be restarted on clarithromycin. When observed on Aug. 15, 2002, she stated she had become “100% better” by Aug. 11, 2002. The patient was continued on clarithromycin at 500 mg b.i.d. and observed serially at weekly intervals on Aug. 22, 2002, Aug. 27, 2002, and Sep. 5, 2002. Her symptoms continued under good control and she stated that she was “doing well” at each observation time. This represented a positive re-challenge result, once again demonstrating the efficacy of clarithromycin on this patient.

Laboratory studies revealed no significant change in ESR, CRP, Interleukin-1, or Interleukin-6 (Table 1).

In summary, this patient demonstrated exacerbation of symptoms off clarithromycin, with resolution and control of symptoms when clarithromycin was restarted. These findings are similar to her clinical history over the past several years with respect to clarithromycin therapy and response of connective tissue symptoms.

### Example 3

A 65 year-old male patient has severe ditTuse generalized chondrocalcinosis leading to pseudorheumatoid pseudogout syndrome. Given the intensity of his chronic synovitis and the lack of response to steroids as well as NSAIDs, he was put on methotrexate, 10 mg/week and infliximab (REMICADE®, Centocor, Inc.), after which he did satisfactorily. The patient had bilateral total knee replacements and, subsequently was taken off the infliximab when he developed a knee infection postoperatively. The patient continued to have diffuse aches and pains and joint swelling. When seen in the office on Mar. 12, 2002, he spontaneously asked whether the medicine he was taking for his pneumonia could have an effect on his arthritis. Apparently he had begun clarithromycin 500 mg b.i.d. for his pneumonia two weeks prior and said he had “never felt as well” with respect to his joint symptoms as he had during the subsequent several days. He continued to feel much improved and asked if he could stay on the antibiotic.

### Example 4

Subsequent to the observations made in Example 3, the patient was re-challenged with clarithromycin, and re-evaluated as to symptomatic efficiency. In parallel with clinical observations, laboratory assessments including erythrocyte sedimentation rate, C-reactive 1 5 protein, and serum interleukins 1 and 6 were measured. The laboratory results are illustrated in Table 2.

On Jun. 15, 2002 the patient from Example 3 underwent a right pneumonectomy for treatment of recurrent lung cancer. During that hospitalization, clarithromycin was discontinued. Five to six days following discontinuation of clarithromycin he noted that his arthritis symptoms had become significantly worse, with increased symptoms particularly related to wrists and elbows. When clarithromycin was begun as antimicrobial therapy related to surgical management, his joints once again improved.

The patient underwent formal re-challenge with clarithromycin on Aug. 19, 2002 (Table 2). At that time his medications included ibuprofen at 400 mg/day, acetaminophen at 500 mg/as needed, prednisone at 5 mg/daily, CELEXA™ (Forest Pharmaceuticals, Inc.) at 300 mg/daily, REMURON® (Organon Inc.) at 7.5 mg/h.s., DURICEF® (Bristol) at 500 mg b.i.d., TIMOPTIC® eye drops (Merck & Co.), and multi-vitamins. DURICEF® had been prescribed for treatment of septic arthritis following total knee replacement performed on May 5, 2001. Clarithromycin at 500 mg b.i.d. was restarted on Aug. 20, 2002. When seen on Aug. 27, 2002 he stated that he had not observed improvement in his symptoms compared to baseline on Aug. 19, 2002. Because of a general increase in symptoms following his pneumonectomy, he was placed on increased anti-inflammatory medication, ibuprofen at 800 mg/day on Aug. 28, 2002. On Sep. 4, 2002, he stated he was “40% better”. Ibuprofen was decreased to 600 mg/day on Sep. 5, 2002. On Sep. 16, 2002, he stated that his symptoms were worse; clarithromycin was discontinued. On Sep. 23, 2002, due to a continued worsening of his symptoms, his prednisone dose was increased to 20 mg/day, and his ibuprofen increased to 800 mg/day. When last observed during his re-challenge study on Oct. 8, 2002 his arthritis symptoms continued active.

Laboratory studies, as illustrated in Table 2, revealed a significant increase in C-reactive protein from a baseline value of 0.85 mg/dl on Aug. 19, 2002 to 3.51 mg/dl on Sep. 23, 2002 off clarithromycin. IL-6 increased from a baseline value of 2.2 pg/ml to 33.1 pg/ml on Sep. 16, 2002, one week following clarithromycin discontinuation. No significant change in IL-1 was noted. And ESR increased from 11 mm at baseline to 37 mm. This re-challenge study suggestively supported the efficacy of clarithromycin in relief of the patient's symptoms. The trial was less definitive, however, given requirements for analgesic and anti-inflammatory therapy for non-rheumatic complaints.

In summary, this patient had a suggestive response to clarithromycin on re-challenge but, as noted, therapeutic requirements for non-rheumatologic problems made interpretation of the study more difficult. The flare of symptoms following clarithromycin discontinuation after pneumonectomy supported a response to clarithromycin.

### Example 5

A 38 year old female was first seen in December 1990 for a history of knee symptoms for the prior year. Both knees were involved. In addition, she had “aches and pains all over” for a long period. Knee swelling occurred off and on. Pertinent history review revealed a history of canker sores, and hair loss during pregnancy. She had a sulfonamide allergy. Examination revealed elbow limitation, limitation of hips, and swelling of knees. Pertinent laboratory studies at that time revealed an ANA (anti-nuclear antibody) titer of 1:40. Rheumatoid factor was also normal. Repeat ANA was 1:80. Subsequent anti-nuclear antibody studies revealed a slightly elevated anti-SS-B. A repeat ANA study of Nov. 21, 2001 revealed 1:80 ANA with the remainder of the panel being within normal limits.

In 1991, it was elected to begin the patient on hydroxychloroquine (PLAQUENIL®, Sanofi-Synthelabo, Inc.) given her low-grade connective tissue symptoms not responding to non-steroidal anti-inflammatory therapy. Hydroxychloroquine was subsequently discontinued approximately nine (9) months later because of weight loss associated with this therapy. Therapy with quinacrine (ATABRINE®, Sanofi-Synthelabo, Inc.), another anti-malarial was substituted; however, this was discontinued approximately one year later since she felt that her skin was “too yellow”, a non-toxic condition associated with drug administration; she was also considering trying to become pregnant again. A trial of hydroxychloroquine was again initiated to see how she would tolerate it. Her symptoms continued with swelling and pain in the knees as well as polyarthralgia elsewhere. Hydroxychloroquine was discontinued when she wanted to attempt getting pregnant again. Therapy most recently consisted of non-steroidal inflammatory agents and physical therapy.

On Feb. 27, 2003, the patient agreed to participate in the clarithromycin study. Pertinent findings at baseline revealed swelling of several small joints of the hands, the knees, and one ankle. The patient's estimate of “pain over the past week” on a visual analogue scale was 67 mm (VAS from 0 to 100 mm); patient global was listed at 30 mm; practitioner patient global, 54 mm. The patient was begun on clarithromycin (BIAXIN®, Abbott Laboratories) 500 mg twice per day. When she was evaluated approximately one (1) month later, Apr. 2, 2003 (“Visit Three”), she stated, “the joint pain became better pretty much right away”. “The knees no longer burn and it is easier to drive.” Side reaction included a metallic taste due to the study medication. On physical examination, there was decreased joint tenderness and swelling as illustrated in Table 3. VAS for pain was now 26 mm, and patient global assessment was 12 mm. Practitioner assessment of patient global was 25 mm. As indicated by Table 3, all parameters showed significant decrease from baseline.

### Example 6

A 47 year-old female patient, was first seen in September 1999. She had a long history of polyarthralgia involving many joints, with main symptoms at that time in the knees. Her past history revealed a migratory arthritis with intermittent episodes over the past 15 years. She often awakened with pain that involves her knees, neck and upper extremity joints. She has had short courses of steroids for acute pain episodes which may occur a number of years apart. She had wrist swelling approximately 1½ years prior. She has used a number of anti-inflammatory agents frequently for therapy. She also had a history of severe cervical spine disc degeneration.

Physical exam revealed slight thickening of the right hand. She was tender along the epicondylar areas of the elbows. The right knee was swollen grade “1−”. Capillary microscopy of the right second and third fingers revealed a slightly irregular pattern with some dropout consistent with early connective disease changes. System review revealed recent canker sores and a history of Raynaud's.

On the basis of the findings, including recurrent episodes of arthritis, Raynaud's, and thickening of the hands, connective tissue disease diagnoses such as Mixed Connective Tissue Disease or Progressive Systemic Sclerosis were considered. Laboratory studies revealed an erythrocyte sedimentation rate of 60 mm per hr. Her ANA was 1:320; remainder of her ANA panel was negative.

Based on these findings, a diagnosis of Undifferentiated Connective Tissue Disease was made. Therapy was initiated with hydroxychloroquine (PLAQUENIL®, Sanofi-Synthelabo, Inc.) and a non-steroidal anti-inflammatory agent. She utilized steroid dose packs periodically for symptomatic relief of her peripheral symptoms as well as her neck symptoms. Hydroxychloroquine was discontinued after approximately six months, since she felt that she did not feel significant improvement.

When last seen, she continued to have migratory joint symptoms. It is of interest that she noted, upon questioning, that when she had once taken ciprofloxacin (CIPRO®, Bayer Corporation) 500 mg twice per day, the pain would be improved. Accordingly, she tried this again on her own for approximately six weeks but did not find it effective at this time. Similarly, she tried amoxicillin, but this was also of no help. On examination, she had symptoms and findings much as before and a diagnosis of Undifferentiated Connective Tissue Disease was made. The patient agreed to undergo a trial with clarithromycin.

Following screening and baseline evaluations, the patient was begun on clarithromycin (BIAXIN®, Abbott Laboratories). At her baseline visit, she noted “aching all over” with an increase in joint pain over the three days prior to the baseline visit. There was no change in her Raynaud's, and her fatigue and sleep deprivation had become worse. Physical examination revealed pain and tenderness in the shoulder and elbow areas, and swelling of the right wrist and several small joints of the hands. The patient's assessment of VAS for pain was 89 mm, and patient global VAS was 63 mm. Practitioner's assessment of patient global VAS was 60 mm.

The patient was seen for re-evaluation one month later on Mar. 25, 2003 (“Visit 3”), for her first recheck visit. In the interim, she had noted increased neck spasms that were treated with methyl-prednisolone by her physician for approximately one week followed by 5 mg of methyl-prednisolone daily for approximately one week.

When seen for her one month evaluation, the patient had been off steroid medication for approximately ten days. The practitioner estimate of patient global status was 31 mm VAS; the patient's assessment of pain was 16 mm VAS, and patient global was 51 mm. Accordingly, she demonstrated improvement from her baseline evaluation. Joint assessment at this one month interval revealed swelling of only one PIP joint as illustrated in Table 4, demonstrating significant resolution of her prior joint findings.

Although the patient had demonstrated improvement from baseline at the one-month visit on clarithromycin, her need to take a two-week course of steroids (recommended by her physician) for severe neck pain was a confounder in the assessment of clarithromycin effect. It is of interest that significant improvement was subsequently noted, however, when she had been off steroids for ten days at the time of the one-month visit. Further assessment of her status was made on Apr. 13, 2003 at which time she was now off steroids for almost a one-month period. She stated that she was continuing to improve; she had fewer spontaneous flares “with hardly any more short hits of migratory polyarthritis.” She rated her pain at VAS 25 mm. Although she still complained of a bad taste, she felt she wished to continue on the trial, given what she felt was an improvement in her symptoms.

Patient was again seen Apr. 21, 2003 (“Visit Four”), for her formal two-month follow-up on clarithromycin. She stated she had no symptoms currently and, on questioning, stated “she felt 80% better.” She reported, “I have no joint pain at all.” Her only side reaction was mild bad taste. VAS for patient pain was 11 mm; patient global 14 mm; and practitioner global 4 mm. She had no joint tenderness or swelling. Accordingly, she has had excellent symptomatic response and plans to continue on medication as per protocol as illustrated in Table 4.

This written description uses examples to disclose the invention, including the best mode, and also to enable any person skilled in the art to make and use the invention. The patentable scope of the invention is defined by the claims, and can include other examples that occur to those skilled in the art. Such other examples are intended to be within the scope of the claims if they have elements that do not differ from the literal language of the claims, or if they include equivalent elements with insubstantial differences from the literal language the claims.

TABLE 1
Example 2 Re-challenge Results for Example 1 Patient

| | | | | IL-1 | IL-6 | CPR | ESR |
|---|---|---|---|---|---|---|---|
| Date | Clarithromycin | Symptoms | Medications | pg/ml | pg/ml | mg/dL | mm/hr |
| Jul. 29, 2002 | Yes | Good Control | Clarithromycin 500 mg/po BID^{a} | ND^{b} | No | <0.3 | 29 |
| | | | Zestril 1 mg/po TID | | Data | | |
| | | | Caltrate w/D/po BID | | | | |
| | | | Unithroid 0.05/mg/po/QD | | | | |
| | | | Trazodone 550 mg @ US | | | | |
| | | | Tylenol 1 gm/pm | | | | |
| | | | Beano prn | | | | |
| | | | Lactaid prn | | | | |
| | | | Multivitamin prn | | | | |
| | | | Biotin forte eye gtts BID | | | | |
| | | | Slow Fe po QD | | | | |
| | | | Quinine 260 mg | | | | |
| | | | Beta Glucan immunition NSC-24 QD (herbal) | _______ | | | |
| Aug. 8, 2002 | No^{d} | Generalized | Same as above. | ND | 2.1 | No | 25 |
| | | joint pain/ | Clarithromycin continued. | | | Data | |
| | | swelling | | | | | |
| Aug. 15, 2002 | Yes | 100% better | Same | ND | 0.9 | <0.3 | 21 |
| | | on Aug. 11, 2002 | | | | | |
| Aug. 22, 2002 | Yes | Doing well | Same | ND | 0.7 | <0.3 | 34 |
| Aug. 27, 2002 | Yes | Doing well | Same | ND | 1.1 | <0.3 | 21 |
| Sep. 5, 2002 | Yes | Doing well | Same | ND | 1 | <0.3 | 34 |

^{a}Clarithromycin discontinued at this visit.
^{b}ND = non-detectable.

TABLE 2
Example 4 Re-challenge Results for Example 3 Patient

| | | | | IL-1 | IL-6 | CPR | ESR |
|---|---|---|---|---|---|---|---|
| Date | Clarithromycin | Symptoms | Medications | pg/ml | pg/ml | mg/dL | mm/hr |
| Aug. 19, 2002 | No | Baseline | Ibuprofen 400 mg/d | ND^{a} | 2.2 | 0.85 | 11 |
| | | | Acetominaphen 500 mg/prn | | | | |
| | | | Prednisone 5 mg/QD | | | | |
| | | | Celexa 30 mg/QD | | | | |
| | | | Remuron 7.5 mg @ HS | | | | |
| | | | Duricef 500 mg/BID | | | | |
| | | | Timoptic eye gtts | | | | |
| | | | Muitivitamin QD | | | | |
| Aug. 20, 2002 | No | No data | Clarithromycin 500/BID begun. | No | No | No | No |
| | | | | Data | Data | Data | Data |
| Aug. 27, 2002 | Yes | No change | Same | ND | 6.8 | 0.74 | 18 |
| Sep. 4, 2002 | Yes | 40% better | Ibuprofen increased to 800 mg/day on Aug. 28, 2002. | ND | 2.5 | 0.78 | 10 |
| Sep. 9, 2002 | Yes | No change | Ibuprofen decreased to 600 mg/day on Sep. 5, 2002. | ND | 4.5 | 1.06 | 11 |
| | | | Clarithromycin discontinued. | | | | |
| Sep. 16, 2002 | No | Worse | Ibuprofen decreased to 400 mg/day on Sep. 13, 2002. | ND | 33.1 | No | No |
| | | | Others without change. | | | Data | Data |
| Sep. 23, 2002 | No | Worse | Prednisone dose increased to 20 mg/day. | ND | 6.4 | 3.51 | 37 |
| | | | Back to Ibuprofen 800 mg/day. | | | | |
| | | | Others without change. | | | | |
| Oct. 8, 2002 | No | Same as | Prednisone 5 mg QD | ND | 1.8 | 0.77 | 10 |
| | | baseline | Others without change. | | | | |

^{a}ND = non-detectable.

TABLE 3
Example 5

| Criteria | Baseline | | Visit Three^{a} | |
|---|---|---|---|---|
| Patient VAS (Pain) | 67 | mm | 26 | mm |
| Patient VAS (Global) | 30 | mm | 12 | mm |
| Practitioner VAS (Global) | 54 | mm | 25 | mm |
| Tender/Painful Joints | 3 | | 0 | |
| Swollen Joints | 6 | | 1 | |

^{a}Patient on clarithromycin therapy for one month.

TABLE 4
Example 6

| Criteria | Baseline | | Visit Three^{a} | | Visit Four^{b} | |
|---|---|---|---|---|---|---|
| Patient VAS (Pain) | 89 | mm | 16 | mm | 11 | mm |
| Patient VAS (Global) | 63 | mm | 51 | mm | 14 | mm |
| Practitioner VAS (Global) | 60 | mm | 31 | mm | 4 | mm |
| Tender/Painful Joints | 3 | | 0 | | 0 | |
| Swollen Joints | 6 | | 1 | | 0 | |

^{a}Patient on clarithromycin therapy for one month.
^{b}Patient on clarithromycin therapy for two months.

I claim:

1. A composition for treating a patient having a connective tissue disease comprising a therapeutically effective amount of a macrolide antibiotic selected from the group consisting of a 14-member macrolide antibiotic, a 15-member macrolide antibiotic, pharmaceutically acceptable derivatives thereof, and combinations thereof

2. The composition of claim 1, wherein the macrolide antibiotic is selected from the group consisting of natural, synthetic, semi-synthetic macrolide antibiotics, and mixtures thereof.

3. The composition of claim 1, wherein the macrolide antibiotic is selected from the group consisting of erythromycin A, clarithromycin, roxithromycin, dirithromycin, HMR 3004, azithromycin, pharmaceutically acceptable derivatives thereof, and combinations thereof.

4. The composition of claim 1, wherein the connective tissue disease is selected from the group consisting of rheumatoid arthritis, psoriatic arthritis, giant cell arteritis, systemic lupus erythematosus, polymyalgia rheumatica, polymyositis, and inflammatory bowel disease.

5. A method for treating a patient having a connective tissue disease comprising administering to the patient a therapeutically effective amount of a macrolide antibiotic selected from the group consisting of a 14-member macrolide antibiotic, a 15-member macrolide antibiotic, pharmaceutically acceptable derivatives thereof, and combinations thereof.

6. The method of claim 5, wherein the macrolide antibiotic is selected from the group consisting of natural, synthetic, semi-synthetic macrolide antibiotics, and mixtures thereof.

7. The method of claim 5, wherein the macrolide antibiotic is selected from the group consisting of erythromycin, clarithromycin, roxithiomycin, dirithromycin, HMR 3004, azithromycin, pharmaceutically acceptable derivatives thereof, and combinations thereof.

8. The method of claim 5, wherein the therapeutically effective amount is a normal clinical dose used to treat bacterial infections.

9. The method of claim 5, wherein the therapeutically effective amount is a maximum dose capable of being safely received by the patient.

10. The method of claim 5, wherein the therapeutically effective amount is a minimum clinically effective dose capable of alleviating at least one symptom of the connective tissue disease.

11. The method of claim 5, wherein the therapeutically effective amount is about 0.01 mg/kg to about 1000 mg/kg of the body weight of the patient.

12. The method of claim 5, wherein the therapeutically effective amount is about 0.01 mg/kg to about 500 mg/kg of the body weight of the patient.

13. The method of claim 5, wherein the therapeutically effective amount is about 0.01 mg/kg to about 250 mg/kg of the body weight of the patient.

14. The method of claim 5, wherein the therapeutically effective amount is about 0.01 mg/kg to about 100 mg/kg of the body weight of the patient.

15. The method of claim 5, wherein the therapeutically effective amount is about 0.01 mg/kg to about 25 mg/kg of the body weight of the patient.

16. The method of claim 5, wherein the therapeutically effective amount is between about 1 mg and about 5500 mg per day.

17. The method of claim 16, wherein the therapeutically effective amount is delivered as a single dose per day.

18. The method of claim 16, wherein the therapeutically effective amount is delivered in multiple doses per day.

19. The method of claim 5, wherein the therapeutically effective amount is delivered on a non-daily dosage schedule.

20. The method of claim 5, wherein the daily dosage level varies over an administration cycle.

21. The method of claim 5, wherein the connective tissue disease is selected from the group consisting of rheumatoid arthritis, psoriatic arthritis, giant cell arteritis, systemic lupus erythematosus, polymyalgia rheumatica, polymyositis, inflammatory bowel disease, and combinations thereof.

22. The method of claim 5, wherein the method continues for a period of time sufficient to alleviate at least one symptom of the connective tissue disease.

23. The method of claim 22, wherein the period of time is about three to about six months.

24. The method of claim 22, wherein the period of time is longer than about six months.

25. The method of claim 5, further comprising the step of readministering a therapeutically effective amount of the macrolide antibiotic at an interval as required to obtain and/or maintain the desired alleviation of one or more symptoms of the disease.

26. The method of claim 5, wherein the macrolide antibiotic is administered by a route selected from the group consisting of oral, transdermal, intravenous, intradermal, intraperitoneal, subcutaneous, intramuscular, cerebrospinal, topical, and combinations thereof.

27. The method of claim 5, further comprising administering the macrolide antibiotic in combination with one or more other drugs used to treat connective tissue disease.

28. The method of claim 27, wherein the one or more drugs used to treat connective tissue disease are selected from the group consisting of methotrexate, inflaximab, etanercept, azathioprine, cyclophosphamide, cyclosporine, penicillamine, minocycline, leflunomide, hydroxychloroquine, auranofin, tacrolimus, and mycophenalate mofetil.

29. A method for treating a patient having a connective tissue disease comprising the steps of
(a) administering to a patient having a connective tissue disease, a therapeutically effective amount of a macrolide antibiotic selected from the group consisting of a 14-member macrolide, a 15-member macrolide, pharmaceutically acceptable derivatives thereof, and combinations thereof, and
(b) readministering a therapeutically effective amount of the macrolide antibiotic at an interval as required to obtain and/or maintain a desired alleviation of one or more symptoms of the disease.
